(19) United States
(12) Patent Application Publication
(10) Pub. No.: US 2019/0069810 A1
(43) Pub. Date: Mar. 7, 2019

(54) BODY MOTION SIGNAL PROCESSING APPARATUS

(71) Applicant: SHARP KABUSHIKI KAISHA, Sakai City, Osaka (JP)

(72) Inventor: EIJI SUEMATSU, Sakai City (JP)

(21) Appl. No.: 16/079,830

(22) PCT Filed: Jan. 5, 2017
(86) PCT No.: PCT/JP2017/000089

§ 371 Date: Aug. 24, 2018

(30) Foreign Application Priority Data
Feb. 29, 2016 (JP) 2016-036864

(51) Int. Cl. A61B 5/113; A61B 5/0245; A61B 5/11; A61B 5/08

(52) U.S. Cl. CPC A61B 5/113; A61B 5/0245; A61B 5/72; A61B 5/08; A61B 5/1113; A61B 5/1117

(57) ABSTRACT

Detection of body motion of a subject in a bathroom is accurately performed. A body motion signal processing apparatus (100) includes: a transmission unit that transmits a radio wave to a target; a receiving unit that receives the radio wave reflected by the target; and a detection unit that detects, from a received signal received by the receiving unit, a body motion component signal corresponding to a body motion component that is motion of a body. The target includes a bathroom, and the body motion signal processing apparatus (100) further includes a signal processing unit (40) that, in a case where a subject is in the bathroom, compares amplitude of the body motion component signal detected by the detection unit to amplitude of the body motion component signal that is detected in advance in the bathroom where no subject exists, and outputs a notice based on a comparison result.

### TECHNICAL FIELD

The present invention relates to a body motion signal processing apparatus, and particularly relates to a body motion signal processing apparatus that detects a body motion signal of a subject and performs processing. This application claims priority based on Japanese Patent Application No. 2016-036864 filed in Japan on Feb. 29, 2016. The entire description content of this Japanese Patent Application is incorporated herein by reference.

### BACKGROUND ART

Accidents of particularly aged persons in bathrooms have been increasing and most of the accidents are caused by a health risk (heat shock) that a blood pressure largely fluctuates up and down due to a sudden change in temperature. It is considered that this causes a syncope, a myocardial infarct, a cerebral infarction, an arrhythmia, or the like, and a typical example of the accidents is drowning in a bathtub caused by heat shock.

Against such a problem, for example, in PTL 1 (Japanese Unexamined Patent Application Publication No. 2002-117466), a microwave sensor is installed on a rear side of a ceiling of a bathroom, and when it is determined that there is no body motion of a user on the basis of an output from the microwave sensor, the subject is awoken by sound awaking means or the like.

### CITATION LIST

### Patent Literature

PTL 1: Japanese Unexamined Patent Application Publication No. 2002-117466

### SUMMARY OF INVENTION

### Technical Problem

In a configuration described in PTL 1, the microwave sensor detects not only body motion but also motion of flowing water discharged from a shower or motion of a surface of hot water in a bathtub. For example, it takes about 5 minutes until the motion of the surface of hot water in the bathtub completely stops, and even when the subject is drown, the microwave sensor of PTL 1 detects the drowning as body motion, thus posing a problem that an operation of awaking means such as an alarm is delayed.

An object of the present disclosure is to provide a body motion signal processing apparatus capable of accurately carrying out detection of body motion of a subject in a bathroom.

### Solution to Problem

A body motion signal processing apparatus according to an aspect of the disclosure includes: a transmission unit that transmits a radio wave to a target; a receiving unit that receives the radio wave reflected by the target; and a detection unit that detects, from a received signal received by the receiving unit, a body motion component signal corresponding to a body motion component that is motion of a body, in which the target includes a bathroom, and the body motion signal processing apparatus further includes a signal processing unit that, in a case where a subject is in the bathroom, compares amplitude of the body motion component signal detected by the detection unit to amplitude of the body motion component signal that is detected in advance in the bathroom where no subject exists, and outputs a notice based on a comparison result.

It is preferable that the body motion component includes a heartbeat component that is motion of the body by heartbeat and a breathing component that is motion of the body by breathing, and the signal processing unit includes means for detecting a heart rate and a breathing rate respectively from a heartbeat component signal and a breathing component signal of the body motion component signal, and in a case where the subject is in the bathroom, outputs the notice based on the comparison result of the amplitude, and the heart rate or the breathing rate.

It is preferable that the body motion component signal detected in advance in the bathroom where no subject exists includes a signal of a motion component of shaking of a surface of hot water in a bathtub in the bathroom or a signal of a motion component of flowing water discharged from a shower in the bathroom.

It is preferable that the received signal by the radio wave reflected includes an IQ modulation signal, and the amplitude of the body motion component signal indicates amplitude of a signal in which signals of an I channel and a Q channel of the received signal are combined.

It is preferable that the amplitude of the body motion component signal indicates average amplitude detected by performing sampling of the body motion component signal at a predetermined time interval.

### Advantageous Effects of Invention

According to the disclosure, accurate detection of body motion of a subject in a bathroom is performed.

### BRIEF DESCRIPTION OF DRAWINGS

FIG. 1 schematically illustrates a configuration of a body motion signal processing apparatus 100 according to Embodiment 1.

FIG. 2 illustrates an example of a circuit configuration of a signal processing unit 40 of FIG. 1.

FIG. 3 illustrates another configuration example of the signal processing unit 40 according to Embodiment 1.

FIG. 4 schematically illustrates an example of an aspect of attaching the body motion signal processing apparatus 100 in a bathroom 142.

FIG. 5 schematically illustrates another example of an aspect of attaching the body motion signal processing apparatus 100 in the bathroom 142.

FIG. 6 schematically illustrates a waveform of a body motion signal detected in pre-processing according to Embodiment 1.

FIG. 7 schematically illustrates a waveform of a body motion signal according to Embodiment 2.

FIG. 8 schematically illustrates a waveform of a body motion signal according to Embodiment 3.

FIG. 9 schematically illustrates a waveform of a body motion signal according to Embodiment 4.

FIG. 10 schematically illustrates a waveform of a body motion signal according to Embodiment 4.

FIG. 11 schematically illustrates a waveform of a body motion signal according to Embodiment 4.

FIG. 12 is a flowchart of processing according to Embodiment 5.

### DESCRIPTION OF EMBODIMENTS

A body motion signal processing apparatus of each of embodiments will now be described with reference to drawings. Note that, parts given the same reference sign in the drawings that are referred to have the same function, so that description thereof will not be repeated unless otherwise required.

### [Outline]

The disclosure relates to an apparatus that senses a state of a subject on the basis of a reflection wave obtained by irradiating a target with a microwave (radio wave) in a bathroom.

In the embodiment, a body motion signal processing apparatus 100 includes a transmission unit that transmits a radio wave into a bathroom, a receiving unit that receives the radio wave reflected in the bathroom, and a body motion signal detection unit that detects, from a received signal received by the receiving unit, a body motion signal indicating motion of a body. The body motion signal processing apparatus 100 compares an amplitude value of a signal of a body motion region component of a received signal that is detected in a state where there is no subject in the bathroom to an amplitude value of a body motion signal detected while the subject is taking a bath and outputs a result of the comparison.

Thus, it is possible to accurately detect whether the amplitude of the body motion signal of the subject, which is detected while the subject is taking a bath, becomes the same as the amplitude of the signal of the body motion region component when the subject is not in the bathroom, that is, that body motion is not detected (for example, a case where the subject is drown in a bathtub). The detection is able to be performed without waiting until motion by shaking of a surface of hot water in the bathtub completely stops.

In each embodiment, body motion indicates motion of a body including motion (for example, motion of a nose, a mouth, or a chest), such as beating of a heart (heartbeat) or breathing, that appears in a living body. The body motion signal is a signal obtained by measuring body motion by using a sensor or the like and includes a quantifiable signal such as a waveform.

### Embodiment 1

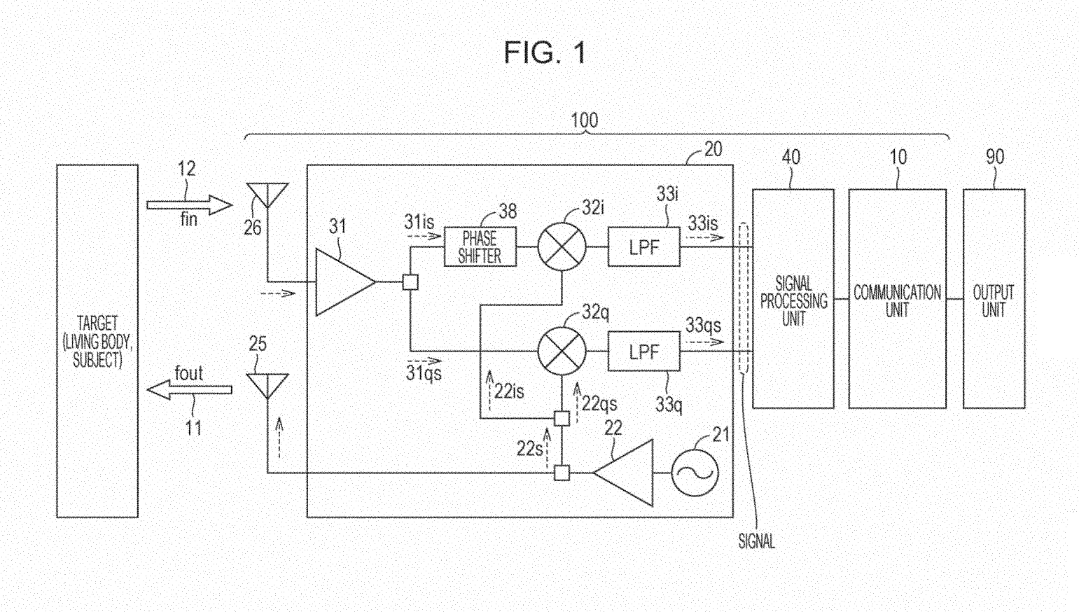

FIG. 1 schematically illustrates a configuration of the body motion signal processing apparatus 100 according to Embodiment 1. With reference to FIG. 1, the body motion signal processing apparatus 100 is configured by including a microwave sensor, and specifically includes a body motion signal detection unit 20, a signal processing unit 40, and a communication unit 10 that communicates with an external device.

### (Configuration of Body Motion Signal Detection Unit 20)

With reference to FIG. 1, the body motion signal detection unit 20 includes an antenna transmission/reception unit and a detection unit. The antenna transmission/reception unit includes a transmitting antenna 25 that transmits a radio wave (transmission signal 11) to a target that includes a subject (living body) and a bathroom and a receiving antenna 26 that receives a radio wave (reflection signal 12) reflected by the target. The detection unit corresponds to a wireless circuit unit of the microwave sensor. In the detection unit, a microwave sinusoidal signal output from an oscillator 21 is amplified by an amplifier 22 and emitted from the transmitting antenna 25 (transmission signal 11), and a radio wave emitted to a space hits and is reflected on the target, for example, a chest part of a living body, a Doppler shift (frequency) is added to a transmitted signal frequency of the reflection signal 12 due to motion (body motion) of the chest including breathing and heartbeat, a frequency and a phase of a transmission wave are modulated by the motion of the body (chest), and the resultant is input to the receiving antenna 26 as the reflection signal 12. Note that, the oscillator 21 outputs a signal with a frequency of a 24 GHz band corresponding to a laser radio wave, for example. Here, an IQ (I: In-Phase, Q: Quadrature) modulation signal is indicated as the signal whose frequency and phase are modulated.

The signal input to the receiving antenna 26 is amplified by a low-noise amplifier 31 and input to mixers 32i and 32q through a phase shifter 38. That is, the signal is split into two signals of an I signal 31is and a Q signal 31qs a phase of which shifts 90 degrees with respect to that of the I signal 31is, and the respective signals are input to the I mixer 32i and the Q mixer 32q. Additionally, a signal 22s that is one of signals obtained by splitting a signal from the oscillator 21 used for a transmission side into two is further split into two signals and the respective signals are input to the I mixer 32i and the Q mixer 32q as an I-side local oscillation signal 22is and a Q-side local oscillation signal 22qs. In Embodiment 1, though the phase of the Q signal 31qs shifts 90 degrees with respect to that of the I signal 31is, a phase of the Q-side local oscillation signal 22qs may shift 90 degrees with respect to that of the I-side local oscillation signal 22is.

An I baseband signal 33is and a Q baseband signal 33qs, which are subjected to frequency down conversion by the I mixer 32i and the Q mixer 32q, are respectively output from a filter 33i and a filter 33q. Each of the I baseband signal 33is and the Q baseband signal 33qs corresponds to a (Doppler frequency) signal wave that has received a Doppler shift due to body motion. A difference between the I baseband signal 33is and the Q baseband signal 33qs is whether or not to pass through a 90-degree phase shifter. Since speed of signals input to the receiving antenna 26 changes over time, phases of the I signal and the Q signal are different by 90 degrees at an instant, and depending on the magnitude of the signal speed and the direction of the signal speed, a phase relationship between the I baseband signal and the Q baseband signal that are output from the filter 33i and the filter 33q is not constant but continuously changes over time. The I baseband signal 33is and the Q baseband signal 33qs are output to the signal processing unit 40. As described above, the detection unit obtains the body motion signal by detecting (extracting) a Doppler shift component included in a reflection wave.

The signal processing unit 40 performs analog signal processing (including AD (analog-to-digital) conversion) and digital signal processing by a DSP (Digital Signal Processor) for the I baseband signal 33is and the Q baseband signal 33qs. The processed signals are transmitted to an output unit 90 through the communication unit 10 in a wireless or wired manner. The output unit 90 includes a display, a speaker, and the like and is controlled by a signal received from the communication unit 10. Thereby, the output unit 90 outputs information (such as an image or sound) according to the received signal.

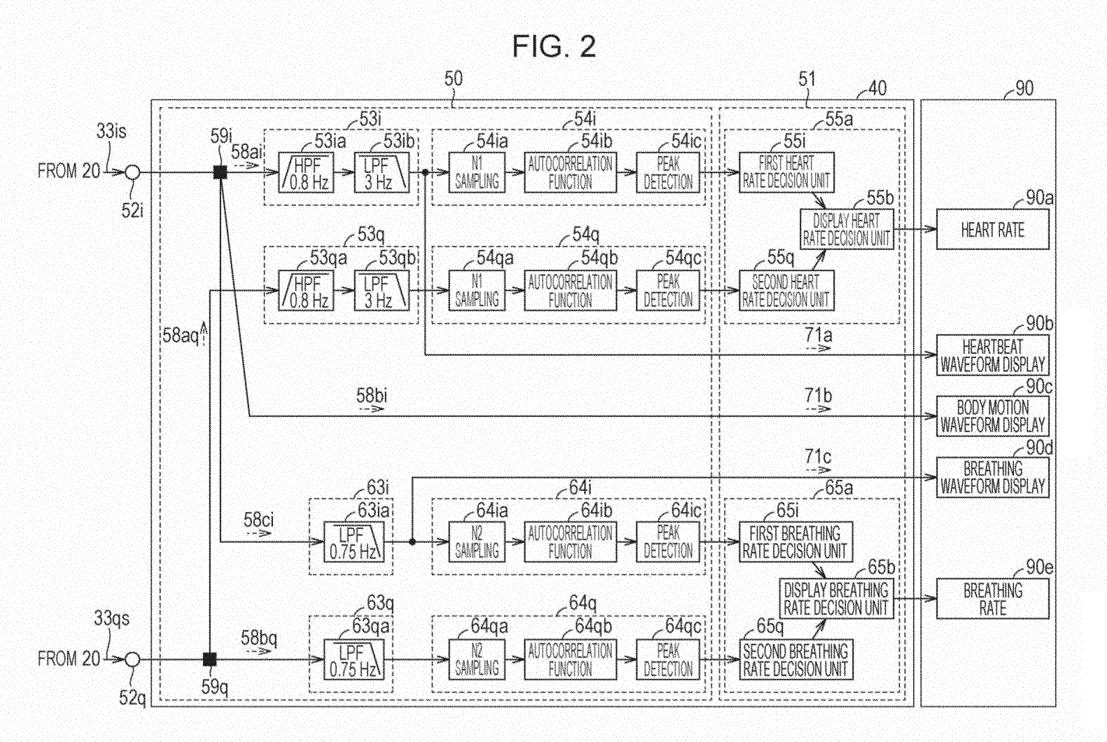

Note that, the signal processing unit 40 may have any of a configuration realized by a program executed by a processor as described above, a configuration realized by a hardware circuit of FIG. 2 described below, and a configuration realized by a combination of the hardware circuit and the program.

### (Example of Configuration of Signal Processing Unit 40)

FIG. 2 illustrates an example of a circuit configuration of the signal processing unit 40 of FIG. 1. With reference to FIG. 2, the signal processing unit 40 includes a conversion unit 50 that converts an analog biological signal mainly input from the body motion signal detection unit 20 into a digital signal (hereinafter, referred to as data), and an information acquisition unit 51 that calculates the converted data to acquire body motion information including biological information (such as a breathing rate or a heart rate) and outputs the body motion information to the communication unit 10. In Embodiment 1, the body motion information may include a waveform of the signal of the body motion region component detected from the reflection signal 12.

The signal processing unit 40 inputs the signal 33is (body motion signal) from the body motion signal detection unit 20 through an input node 52i and inputs the signal 33qs (body motion signal) through an input node 52q.

The signal processing unit 40 includes a distributor 59i that receives the input signal 33is and splits the input signal 33is into first to third I digital signals 58ai, 58bi, and 58ci for outputting, and a distributor 59q that receives the input signal 33qs and splits the input signal 33qs into first and second Q digital signals 58aq and 58bq for outputting. The signal processing unit 40 further includes first and second heartbeat signal extraction units 53i and 53q, first and second breathing signal extraction units 63i and 63q, first and second heartbeat autocorrelation function processing units 54i and 54q, and first and second breathing autocorrelation function processing units 64i and 64q.

The first heartbeat signal extraction unit 53i has a HPF (High Pass Filter) 53ia and a LPF 53ib that receive an input of the I digital signal 58ai and have a filter constant for extracting a signal (heartbeat waveform signal 71a) of a heartbeat component superimposed on the input signal. Similarly, the second heartbeat signal extraction unit 53q has a HPF 53qa and a LPF 53qb that receive an input of the Q digital signal 58aq and have a filter constant for extracting a signal of a heartbeat component superimposed on the input signal.

The first breathing signal extraction unit 63i has a LPF 63ia that receives an input of the I digital signal 58ci and has a filter constant for extracting a signal (breathing waveform signal 71c) of a breathing component superimposed on the input signal. Similarly, the second breathing signal extraction unit 63q has a LPF 63qb that receives an input of the Q digital signal 58bq and has a filter constant for extracting a signal of a breathing component superimposed on the input signal.

The first heartbeat autocorrelation function processing unit 54i has a sampling processing unit 54ia that has a given sampling frequency N1 for converting the heartbeat waveform signal output from the first heartbeat signal extraction unit 53i into digital data, a first heartbeat autocorrelation function calculation unit 54ib, and a peak detection unit 54ic that detects a peak value of a sampling value. The second heartbeat autocorrelation function processing unit 54q has a sampling processing unit 54qa that has a given sampling frequency N1 for converting the heartbeat waveform signal output from the second heartbeat signal extraction unit 53q into digital data, a second heartbeat autocorrelation function calculation unit 54qb, and a peak detection unit 54qc that detects a peak value of a sampling value.

The first breathing autocorrelation function processing unit 64i has a sampling processing unit 54ia that has a given sampling frequency N2 for converting the breathing waveform signal output from the first breathing signal extraction unit 63i into digital data, a first breathing autocorrelation function calculation unit 64ib, and a peak detection unit 64ic that detects a peak value of a sampling value. The second breathing autocorrelation function processing unit 64q has a sampling processing unit 64qa that has a given sampling frequency N2 for converting the breathing waveform signal output from the second breathing signal extraction unit 63q into digital data, a second breathing autocorrelation function calculation unit 64qb, and a peak detection unit 64qc that detects a peak value of a sampling value.

The information acquisition unit 51 has a heart rate decision unit 55a and a breathing rate decision unit 65a. The heart rate decision unit 55a includes first and second heart rate decision units 55i and 55q and a display heart rate decision unit 55b. The breathing rate decision unit 65a has first and second breathing rate decision units 65i and 65q and a display breathing rate decision unit 65b.

The first heart rate decision unit 55i calculates, for example, a representative value of a plurality of (M) peak values that are successively detected by the peak detection unit 54ic. Similarly, the second heart rate decision unit 55q calculates a representative value of a plurality of (M) peak values that are successively detected by the peak detection unit 54qc. The first breathing rate decision unit 65i calculates a representative value of a plurality of (M) peak values that are successively detected by the peak detection unit 64ic. Similarly, the second breathing rate decision unit 65q calculates a representative value of a plurality of (M) peak values that are successively detected by the peak detection unit 64qc. Each of the representative values described above includes a median, a maximum value, a minimum value, a mode, or the like of M values, for example.

The display heart rate decision unit 55b performs given calculation of the representative values from the first and second heart rate decision units 55i and 55q and outputs a value as a result of the calculation to the output unit 90 as display data of a heart rate 90a. Similarly, the display breathing rate decision unit 65b performs given calculation of the representative values from the first and second breathing rate decision units 65i and 65q and outputs a value as a result of the calculation to the output unit 90 as display data of a breathing rate 90e. To the output unit 90, the heartbeat waveform signal 71a that is an output signal from the first heartbeat signal extraction unit 53i, a body motion waveform signal 71b that is one of signals split by the distributor 59i, and the breathing waveform signal 71c that is an output signal from the first breathing signal extraction unit 63i are provided.

The given calculation carried out by the display heart rate decision unit 55b includes calculation processing of an average value of the representative values of the heart rates that are decided by the first and second heart rate decision units 55i and 55q, but a type of the calculation of the representative values is not limited to the calculation of the average value. For example, in a case where a difference between two heart rates exceeds a threshold, the heart rate that is closer to a predetermined normal value may be decided as the heart rate 90a. Alternatively, instead of the heart rate 90a or with the heart rate 90a, data of error display may be output. The display breathing rate decision unit 65b also performs similar calculation for the breathing rate 90e.

In the body motion signal detection unit 20 and the signal processing unit 40 described above, the reflection signal 12 (received signal) is subjected to signal processing by dividing into a real part and an imaginary part, and body motion information including biological information is acquired by using processing results of both the real part and the imaginary part. Thereby, even in an environment where a signal of a body motion region component is able to be extracted only from the real part (only from the imaginary part) depending on a use environment of the body motion signal processing apparatus 100, the body motion information is able to be acquired.

### (Another Configuration of Signal Processing Unit 40)

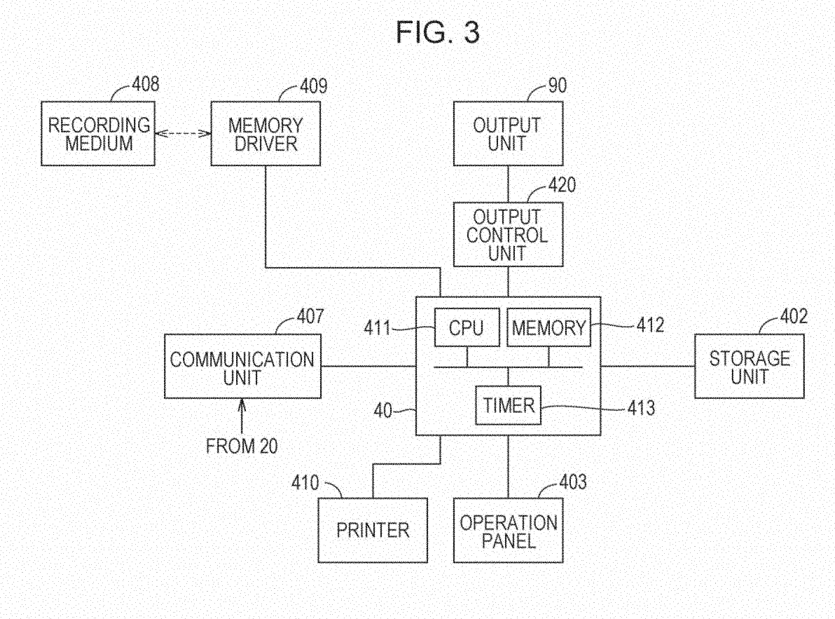

FIG. 3 illustrates another configuration example of the signal processing unit 40 according to Embodiment 1. FIG. 3 illustrates a configuration including a processor that corresponds to the signal processing unit 40. With reference to FIG. 3, the signal processing unit 40 includes a CPU (Central Processing Unit) 111, a memory 412, and a timer 413. The signal processing unit 40 connects a storage unit 402 that stores a program, data, and the like, an operation panel 403 that receives an input to the body motion signal processing apparatus 100 by a user, a communication unit 407 that is used for communication with an external apparatus including the body motion signal detection unit 20, a memory driver 409 to which a recording medium 408 is detachably attached from outside and reads or writes data from or in the recording medium 408 that is attached, a printer 410, and an output control unit 420 that controls the output unit 90. Note that, a display (not illustrated) of the output unit 90 and the operation panel 403 may be integrally constituted to be provided as a tablet device.

### (Attaching Aspect in Bathroom)

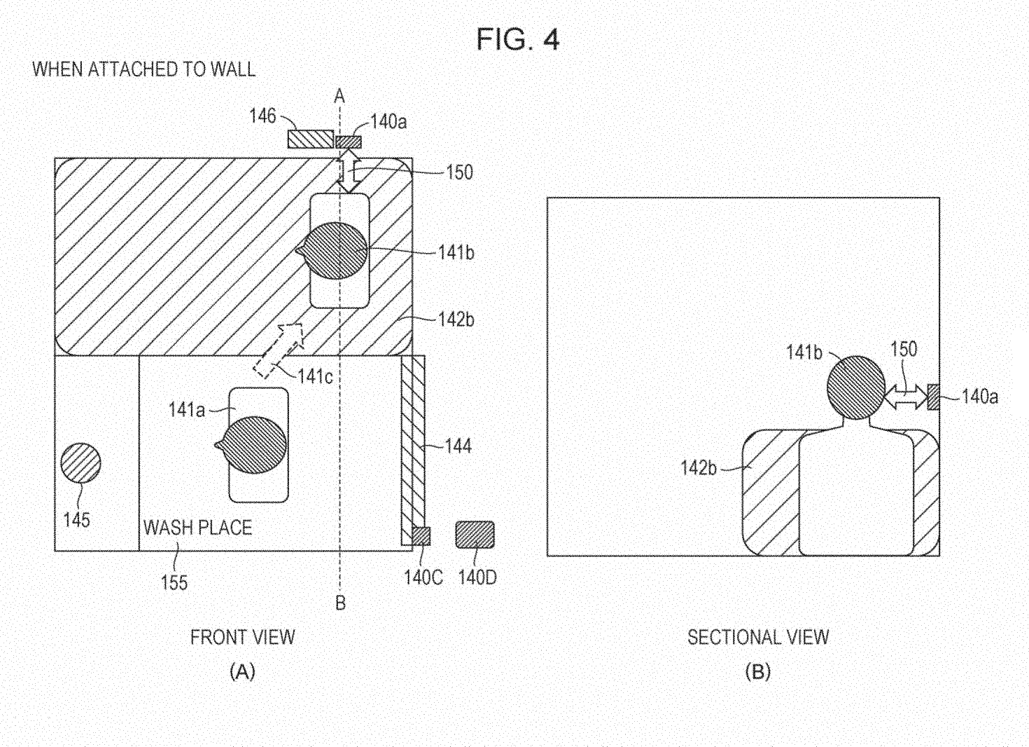

FIG. 4 schematically illustrates an example of an aspect of attaching the body motion signal processing apparatus 100 in a bathroom 142. In FIG. 4(A), a moving direction 141c of a subject 141a from a wash place 155 to a bathtub 142b is indicated. A direction of a transmission/reception radio wave from a microwave sensor 140a is denoted by a sign 150. In the bathroom 142, a door 144 that is opened or closed freely, a door sensor 140C that detects opening or closing of the door 144, and a lighting device 145 are attached. An awaking portion/alarming device 140D that outputs a notice based on a sensing result for a subject in the bathroom 142 is provided outside the bathroom 142. For example, a magnet sensor is able to be used for the door sensor 140C. FIG. 4(B) schematically illustrates a sectional view taken along a broken line A-B of FIG. 4(A).

With reference to FIG. 4, description will be given for a system in which the microwave sensor 140a is installed in the bathroom 142 and body motion of a subject is sensed on the basis of the reflection signal 12 obtained by radiating a microwave into the bathroom 142. In FIG. 4, the microwave sensor 140a corresponding to the body motion signal detection unit 20 is attached to a wall of the bathtub 142b of the bathroom 142, and power is desirably fed from a controller 146 of a hot-water supply system to the sensor 140a. The bathroom 142 has a normal area of two tatami mats, for example.

The microwave sensor 140a is attached around the controller 146, and, in Embodiment 1, while the heartbeat component is sensed as the heart rate and the breathing rate by a direct wave and an indirect wave of the radio wave in the wash place 155, motion of the heartbeat component and the breathing component are sensed by the direct wave in the bathtub 142b. That is, since the bathroom 142 has an area of about two tatami mats, on a wall or the like of the bathroom 142, a reflection component of the radio wave of 24 GHz is large and sensing is performed not only by the direct wave but also by a reflection wave, so that sensing is able to be performed even in a place where the radio wave is not directly sighted.

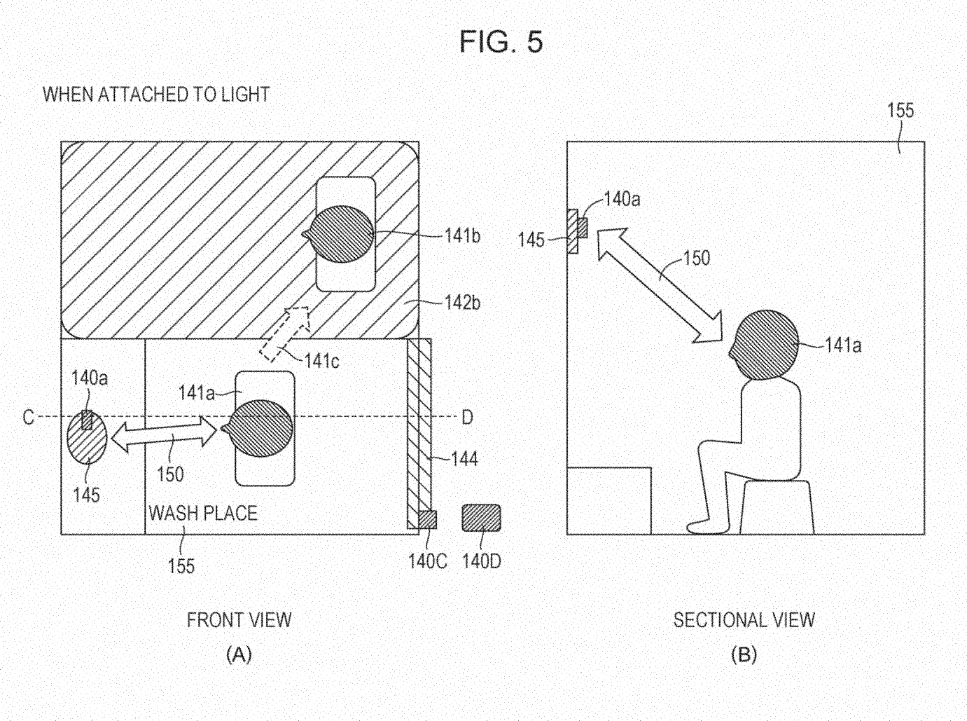

FIG. 5 schematically illustrates another example of an aspect of attaching the body motion signal processing apparatus 100 in the bathroom 142. The microwave sensor 140a is not limited to the aspect of being attached to the controller 146 of the hot-water supply system as in FIG. 4, and may be attached to the lighting device 145 as in FIG. 5(A). FIG. 5(B) schematically illustrates a sectional view taken along a broken line C-D of FIG. 5(A).

### (Pre-Processing)

A case where the subject 141a enters the bathroom 142 and washes his or her body in the wash place 155, and then gets into hot water of the bathtub 142b, and thereafter, leaves the bathtub 142b and exits from the bathroom 142 will be described.

For sensing by the microwave sensor 140a installed in the bathroom 142, pre-processing is performed as a preparatory step. In the pre-processing, the microwave sensor 140a radiates a radio wave in a state where no person (subject 141a) is in the bathroom 142 and receives the reflection signal 12 including a signal indicating motion (shaking) of a surface of hot water in the bathtub 142b, and the signal processing unit 40 detects an amplitude value of a waveform of a frequency band component (hereinafter, also referred to as a body motion region component) of body motion of the received signal. The motion of the surface of hot water may be a representative value of amplitude values obtained until the motion is stopped after a person disappears or a representative value of amplitude values obtained 1.5 minutes to 1 minute before the motion is stopped, for example.

According to an experiment by an inventor, in a case of the bathtub 142b that is normal, it takes about 5 minutes until wave motion of the surface of hot water due to shaking thereof is brought into a stationary state. In view of an experiment result, in the pre-processing, the signal processing unit 40 performs averaging every 100 samples and every 2 seconds with respect to absolute values of amplitude of body motion region components subjected to sampling for 20 milliseconds and further detects amplitude values for about 5 minutes until the surface of hot water is brought into the stationary state after the surface of hot water starts to shake. In Embodiment 1, amplitude values obtained 1.5 minutes to 1 minute before the motion of the surface of hot water is stopped are subjected to sampling. The signal processing unit 40 calculates a representative value of the amplitude values subjected to the sampling and decides the calculated amplitude value (for example, amplitude value=750) as a first threshold 107. The signal processing unit 40 (CPU 411) stores the first threshold 107 in the storage unit 402. Note that, the representative value is acquired by calculating, for example, an average value, a mode, a median, or the like of the amplitude values obtained 1.5 minutes to 1 minute before, and average amplitude is used here.

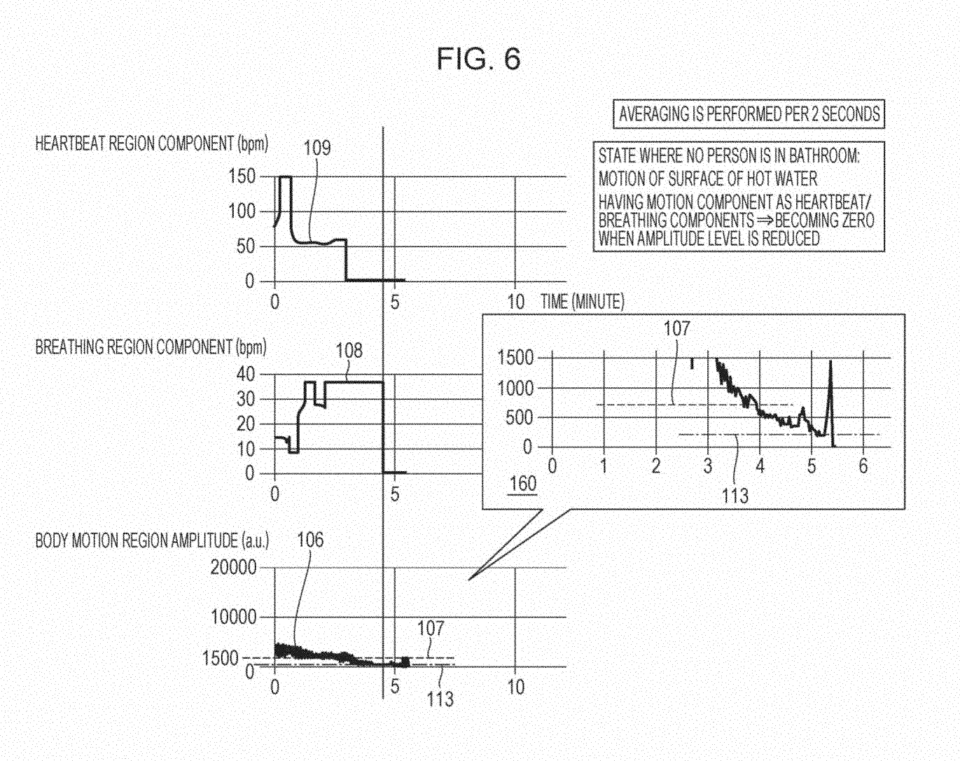

FIG. 6 schematically illustrates a signal of a body motion region component detected in the pre-processing according to Embodiment 1. In FIG. 6, a horizontal axis indicates a time (minute) and a vertical axis indicates voltage amplitude. Specifically, values obtained by performing digital conversion for 0 V to 3 V into 0 to 30000 in 16-bits are assigned as any unit (a.u.) in the vertical axis. In FIG. 6, waveforms of a body motion region component 106, a breathing region component 108, and a heartbeat region component 109 that are included in the reflection signal 12 from motion of the surface of hot water are indicated as measurement signals by the experiment.

### (Processing of Amplitude)

In Embodiment 1, though amplitude of a waveform (hereinafter, also referred to as a waveform of a body motion signal) of a body motion region component that is a reflection component from a subject is average body motion amplitude of absolute values for 2 seconds described above of an I channel, the average body motion amplitude may be average body motion amplitude in which the I channel and a Q channel are combined. In this case, since the I channel has cosine body motion amplitude and the Q channel has sine body motion amplitude, a distance between a person and the microwave sensor 140a causes a zero point every wavelength/2 (for example, about 6 mm in the case of 24 GHz), so that a null point of the cosine and the sine is compensated by combining both the channels by obtaining a sum of root-mean-square values of the I channel and the Q channel, thus making it possible to detect the body motion amplitude more stably.

With reference to FIG. 6, the amplitude of the signal of a wave (body motion region component 106) of the surface of hot water indicates a change in the average amplitude every 2 seconds until the wave of the surface of hot water disappears and the motion is brought into the stationary state after the subject 141a bathing himself or herself in the bathtub 142b stands up from the surface of hot water in the bathtub 142b so that the surface of hot water is stirred and the subject 141a exits from the bathroom 142. The average amplitude takes about 5 minutes until the surface of hot water is stirred and is then brought into the stationary state. In Embodiment 1, the first threshold 107 is acquired on the basis of the amplitude 1.5 minutes to 1 minute before the motion of the surface of hot water is stopped as described above.

In Embodiment 1, the first threshold 107 indicates a value of 750. In this case, the value after the motion of the surface of hot water is completely stopped corresponds to a noise level 113 of the microwave sensor 140a and indicates a value of about 400 in Embodiment 1. The signal processing unit 40 (CPU 411) detects a value of the noise level 113 and stores the value in the storage unit 402.

It is necessary to confirm that there is a somewhat great amplitude level difference between the first threshold 107 (=750) and the noise level 113 (=about 400).

Note that, the body motion amplitude value may be standardized with the first threshold 107. A second threshold 123 described below may be standardized with the noise level 113 of the amplitude value. Since each of the thresholds varies depending on a place where the microwave sensor 140a is installed, the pre-processing needs to be performed after the microwave sensor 140a is installed.

### (Pre-Processing of Signal Components of Heartbeat and Breathing)

In the pre-processing of Embodiment 1, signal components of bands corresponding to a heart rate and a breathing rate that are included in a reflection wave are respectively referred to as a heartbeat region component and a breathing region component. When the motion by shaking of the surface of hot water is displayed with a frequency component of 0.8 Hz to 4 Hz that is a frequency component of the heartbeat region and a frequency component of 0.1 Hz to 0.8 Hz that is a frequency component corresponding to breathing, since there is no subject 141a (person) in the bathroom 142, the motion of the surface of hot water is relatively moderate motion with about 1 Hz (60 bpm: beat per minute) and has a wave closer to monotonous simple vibration.

Thus, in a case where a person gets into the bathtub 142b, even when there is almost no body motion of the person, it is possible to detect quick movement with 90 bpm (1.5 Hz) or more for 1 minute or slow breathing motion with about 24 bpm (0.4 Hz) or less for 1 minute.

The signal processing unit 40 desirably generates, on the basis of a body motion signal that is detected, a caution signal to be output when a state of the subject 141a in the bathtub 142b is determined as a caution state from the body motion signal. That is, the signal processing unit 40 generates a first caution signal on the basis of the body motion signal, and when determining that the state is the aforementioned caution state on the basis of the heart rate by the heartbeat region component or the breathing rate by the breathing region component, generates a second caution signal on the basis of the breathing rate or the heart rate.

In Embodiment 1, though the threshold is set to each of the signal of the heartbeat region component and the signal of the breathing region component, it may be configured in such a manner that the signal threshold is increased to a level of wave surface motion of the surface of hot water and controlled, and when excessively small motion reaches, for example, the level of wave surface motion, display with bpm that is display of heartbeat or breathing becomes zero (the breathing rate or the heart rate is zero). That is, in a state where the person bathes himself or herself in the bathtub 142b and when the person hardly moves, the signal processing unit 40 may set zero as the breathing rate or the heart rate and output a caution signal constituted by only the first caution signal based on only the body motion signal.

### Embodiment 2

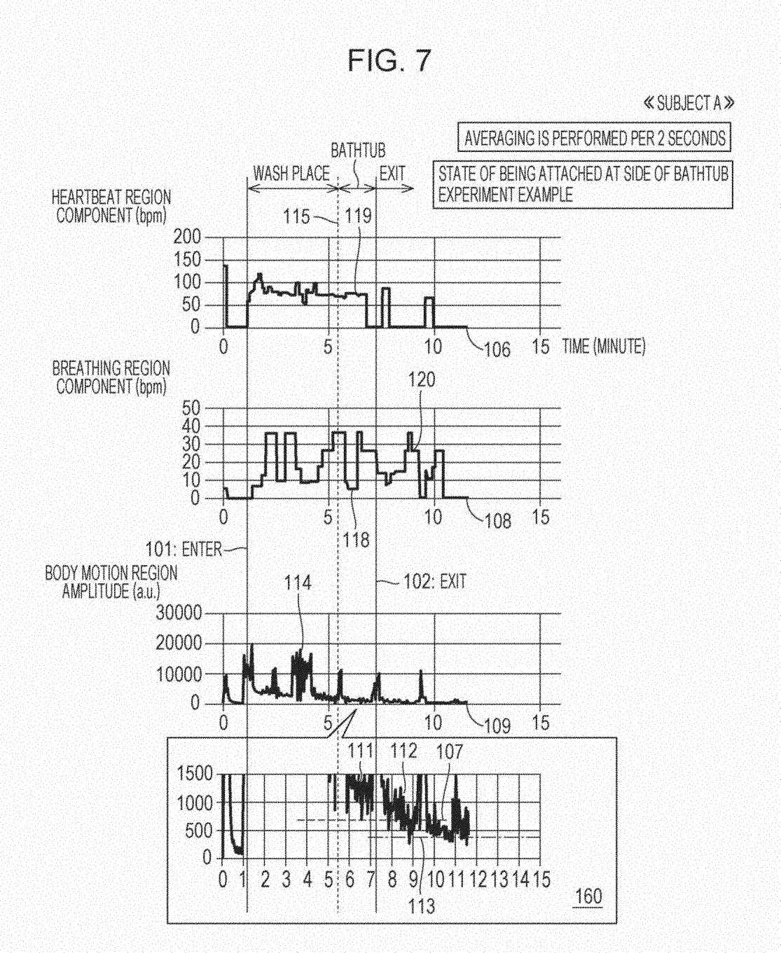

In Embodiment 2, description will be given for detection of body motion, heartbeat, and breathing of the subject 141a by the microwave sensor 140a in a case where the microwave sensor 140a is attached to a wall at a side of the bathtub 142b as illustrated in FIG. 4. FIG. 7 schematically illustrates a waveform of a body motion signal by an experiment according to Embodiment 2. In FIG. 7, a horizontal axis indicates a time (minute) and a vertical axis indicates voltage amplitude. Specifically, values obtained by performing digital conversion for 0 V to 3 V into 0 to 30000 in 16-bits are assigned as any unit (a.u.) in the vertical axis. In FIG. 7, waveforms of the body motion region component 106, the breathing region component 108, and the heartbeat region component 109 that are included in the reflection signal 12 from motion of the surface of hot water are indicated.

In the embodiment, an example of performing sensing by assuming that a subject is a healthy person (referred to as a subject A) who does not normally sleep while taking a bath will be described. In such a case, a threshold having a relatively large value is set (stored in the storage unit 402) to each of the heartbeat region component and the breathing region component, and the threshold is set so that the heartbeat region component is zero even when an amplitude value of the waveform of the body motion signal is substantially the same as that of the waveform indicating motion of the surface of hot water.

With reference to FIG. 7, indicated is a state where sensing is performed for body motion (BM: Body Motion), a heart rate (Heart), and a breathing rate (Breath) in a case where the subject A enters the bathroom 142 at a time 101, washes his or her body in the wash place 155 for a few minutes, and then gets into the bathtub 142b at a time 115, and after bathing himself or herself in the bathtub 142b for about 2 minutes, exits from the bathroom 142 at a time 102. Here, the body motion (BM) is an average value per 2 seconds (of 100 samples of 20 milliseconds).

In this case, a detection waveform of the microwave sensor 140a is indicated in FIG. 7. With reference to FIG. 7, while the subject A washes his or her body in a standing or seating posture in the wash place 155, the body motion changes largely as indicated with a waveform 114 of the body motion signal and a waveform 119 of the heartbeat region component and a waveform 118 of the breathing region component do not indicate correct heart rate and breathing rate. On the other hand, when the subject A gets into the bathtub 142b, a pulse rate by motion of an artery above a neck and motion of breathing in neck and face portions are detected as indicated with the waveforms 119 and 118 and the heart rate and the breathing rate are able to be detected. When the subject A gets into the bathtub 142b and remains relatively still in the seating posture as described above, the waveforms 119 and 118 roughly indicate the heart rate and the breathing rate. In FIG. 7, when the subject A bathing himself or herself in the bathtub 142b moves his or her body such as his or her hand in the bathtub 142b (that is, in hot water), the breathing region component indicated by the waveform 118 tends to increase.

Normally, an action of the subject A at the time 101 of entrance, the time 115 of getting into the bathtub 142b, and the time 102 of exit involve an action of “stand to seat/seat to stand” in the bathroom 142 and average amplitude of the body motion indicates large motion exceeding a level of 10000 as indicated with a waveform 111 of FIG. 7, for example. In the waveform 114 indicating an action of the subject A in the wash place 155, the average amplitude of body motion has a level of about 3000 to 10000, for example, because there is motion of the hand or the like.

On the other hand, when the subject A gets into the bathtub 142b and moves his or her hand slowly, the amplitude of the body motion is relatively large, and when a case where the body motion (body motion indicated with the waveform 111) while the subject A remains still becomes equal to or less than the first threshold 107 of the motion level of the surface of hot water that is detected in the pre-processing occurs, for example, multiple times, in Embodiment 2, for example, three times successively, the signal processing unit 40 outputs a caution signal. In the case of the subject A, while he or she is taking a bath, the motion does not become less than motion of the surface of hot water and the heart rate or the breathing rate does not fall to zero, and the subject A exits from the bathroom 142 at the time 102. In the case of FIG. 7, it is indicated that the subject A finishes taking a bath without trouble.

Note that, the surface of hot water shakes for about 5 minutes after the subject A exits from the bathroom 142 (when a lid for the bath is not put after exit), and the surface of hot water continuously shakes as indicated with the waveform 112 for about this 5 minutes, but after 5 minutes have lapsed, the shaking is reduced and the amplitude value of the signal of the body motion region component of the microwave sensor 140a is almost the noise (level) 113.

### Embodiment 3

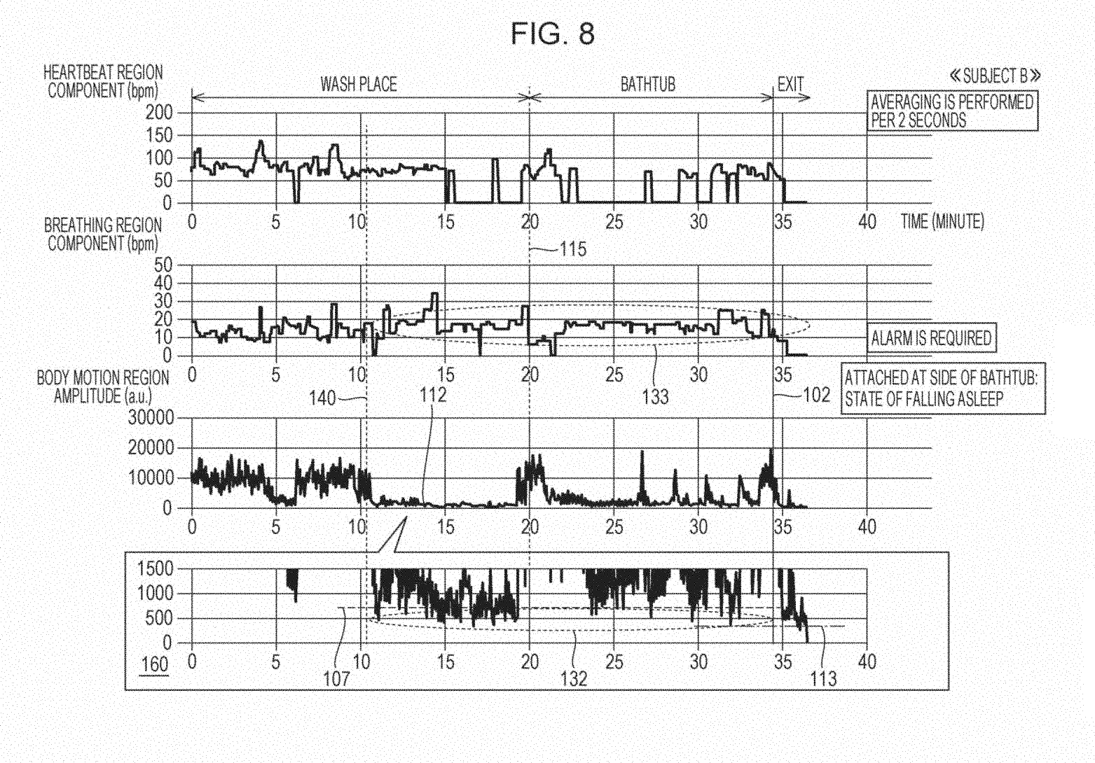

Next, an experiment example when a healthy person (subject B) falls asleep in the bathroom 142 will be described. A case of falling asleep in the bathroom 142 has high possibility of danger and is treated as a danger matter in the bathroom. FIG. 8 schematically illustrates a waveform of a body motion signal according to Embodiment 3. In FIG. 8, a horizontal axis indicates a time (minute) and a vertical axis indicates voltage amplitude. Specifically, values obtained by performing digital conversion for 0 V to 3 V into 0 to 30000 in 16-bits are assigned as any unit (a.u.) in the vertical axis. In FIG. 8, waveforms of the body motion region component 106, the breathing region component 108, and the heartbeat region component 109 that are included in the reflection signal 12 are indicated.

Also in Embodiment 3, the aforementioned signal thresholds of heartbeat and breathing are relatively large and setting is performed so that the heartbeat component is zero even when the amplitude of the body motion has substantially the same level as that of the surface of hot water.

In FIG. 8, in a case where the subject B falls asleep when being in the wash place 155, that is, at a time 140 12 minutes later after start of measurement, the amplitude of the waveform 112 of the body motion region component is suddenly reduced, and 15 minutes later, the body motion becomes completely small and the waveform 112 of the body motion is made as small as the amplitude of a waveform 132 indicating the motion of the surface of hot water. At this time, the heartbeat falls to zero because the signal thresholds are set so that the heart rate becomes zero when the amplitude value of the signal of the body motion region component has substantially the same level as that of the amplitude value of the waveform of the motion of the surface of hot water, but the waveform 133 of breathing is able to be monitored at all times.

Though the subject B wakes up from a sleep once and bathes himself or herself in the bathtub 142b when 19 to 20 minutes have lapsed after start of the measurement, but 24 minutes later, the subject B falls asleep in the bathtub 142b. In such a case, the amplitude value of the body motion region component has the same level (that is, the first threshold 107) as that of the amplitude value of the waveform indicating shaking of the surface of hot water. In this case, though the heart rate sometimes falls to zero for a while, the breathing region component is able to be detected at all times as indicated with the waveform 133.

In Embodiment 3, in a case where the subject B falls asleep, the amplitude value of the body motion signal reaches 750 as the value of the first threshold 107 multiple times (for example, three or more times in Embodiment 3) successively, the signal processing unit 40 outputs a caution signal, activates awaking means with alarm sound of a speaker or buzzer of the output unit 90 or activates communication means, and transmits a notice of caution to a terminal or the like of a third person through communication and causes the terminal to perform output. This makes it possible for the third person to find and save the subject B before he or she completely falls asleep (in a case of an aged person, before falling asleep and drown).

Here, the output of the caution signal is generally able to be realized when the signal processing unit 40 compares a signal level input from the body motion signal detection unit 20 to the first threshold 107 and outputs a control signal to switch a switch of an alarm of the output unit 90 from off to on in accordance with a result of the comparison, but a method for outputting the alarm is not limited thereto.

Further, it is also possible to perform the output of the caution signal in a stepwise manner and set activation of the awaking means and activation of the communication means for alarming in a stepwise manner, and an example thereof is indicated below.

(1) When the amplitude value of the signal of the body motion region component exceeds 750 and the subject is breathing and his or her heart is beating, no caution signal is provided.

(2) When the amplitude value of the signal of the body motion region component exceeds 750 and the subject is breathing but his or her heart is not beating, a caution signal is provided. For example, an intermittent alarm or rhythm sound for awaking is provided.

(3) When the amplitude value of the signal of the body motion region component is 750 or less and the subject is breathing and his or her heart is beating, a caution signal is provided. For example, a consecutive alarm for awaking is provided.

(4) When the amplitude value of the signal of the body motion region component is 750 or less and the subject is breathing but his or her heart rate is zero, a caution signal is provided. An emergency alarm and a notice are provided for awaking and saving.

(5) When the amplitude value of the signal of the body motion region component is 750 or less and the subject is not breathing and his or her heart rate is zero, a caution signal is provided. An emergency alarm and a notice are provided for awaking and saving.

By outputting the caution signal described above, it is possible to activate the awaking means and the communication means to carry out awaking and saving.

Here, as the average body motion heartbeat, the body motion of the IQ is obtained with an average value of 100 samples for 2 seconds, that is, at a sampling rate of 20 milliseconds per one sample, but an average value (per 0.2 second) of 10 samples or an average value (per 1 second) of 50 samples may be used. The shortening improves detection of breakthrough motion or the like and real-time property.

In addition, by detection of the reflection signal 12 by the body motion signal detection unit 20 with use of the microwave sensor 140a and signal processing of the signal processing unit 40, entrance of a subject into the bathroom 142 is able to be determined on the basis of signals of the body motion region component, the heartbeat region component, and the breathing region component. In a case of exit from the bathroom 142, though it is possible to determine whether the subject dies in the bathroom 142 or already exits from the bathroom 142 on the basis of the first threshold 107 (amplitude value=750) as in (1) to (4) described above, in order to perform determination more reliably, it may be configured in such a manner that a door sensor 140C is installed to detect a change from an open state to a close state or from a close state to an open state of the door 144 of the bathroom 142 and the signal processing unit 40 determines entrance of the subject into the bathroom 142 and exit from the bathroom 142 on the basis of a detection output of the door sensor 140C and a detection signal of the microwave sensor 140a.

### Embodiment 4

Description of Embodiment 4 will be given mainly for a difference from Embodiments 1 to 3. In Embodiment 4, the microwave sensor 140a is attached to the lighting device 145 of the bathroom 142 as illustrated in FIG. 5. In this case, it is desired that the microwave sensor 140a is integrally incorporated in the lighting device 145 so that a power source or the like is able to be desirably shared. This makes it possible to also ensure a waterproofing function or the like. In the aspects of attaching the microwave sensor 140a in Embodiments 1 to 3, it is possible to attach the microwave sensor 140a only when the bathroom 142 is newly provided or remodeled, but in the case of Embodiment 4, it is possible to attach the microwave sensor 140a by replacement with a lighting device with the microwave sensor 140a so that the microwave sensor 140a is able to be used also in the existing bathroom 142.

In a case where the microwave sensor 140a is attached to the lighting device 145, as illustrated in FIG. 5, in the bathroom 142 that has a normal area of about two tatami mats, the heartbeat component is sensed as the heart rate and the breathing rate by the direct wave of the radio wave in the wash place 155, but in the bathtub 142b, sensing of the heartbeat component motion and the breathing component is performed by the indirect wave of the radio wave in addition to the direct wave. That is, since the bathroom 142 has an area of about two tatami mats, on the wall or the like of the bathroom 142, a reflection component of the radio wave of 24 GHz is large and sensing is able to be performed not only by the direct wave but also by a reflection wave, so that sensing is able to be performed even in a place where the radio wave is not directly sighted.

### (Pre-Processing)

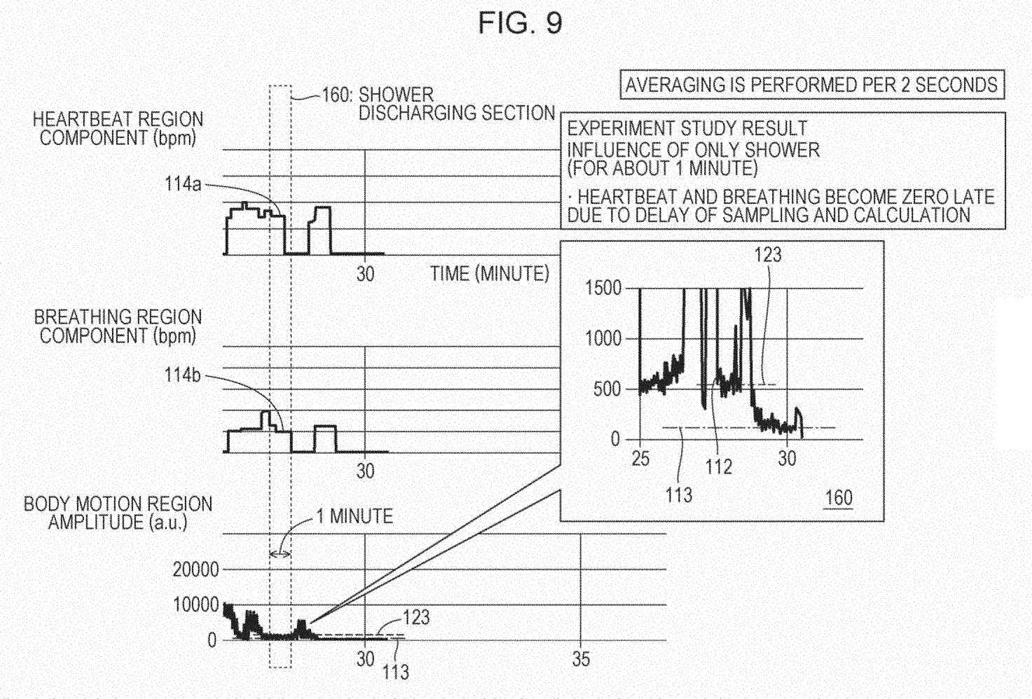

Also in Embodiment 4, similarly to the aforementioned embodiments, the amplitude value by shaking of the surface of hot water in the bathtub 142b is set as the first threshold 107 and the amplitude value of the signal of a reflection wave component (hereinafter, also referred to as a motion component of a shower) in motion of the shower (flowing water discharged from a head of a shower device) is set as the second threshold 123. FIGS. 9, 10 and 11 each schematically illustrates a waveform of a body motion signal according to Embodiment 4. In FIGS. 9 to 11, a horizontal axis indicates a time (minute) and a vertical axis indicates voltage amplitude. Specifically, values obtained by performing digital conversion for 0 V to 3 V into 0 to 30000 in 16-bits are assigned as any unit (a.u.) in the vertical axis. In FIGS. 9 to 11, waveforms of the body motion region component 106, the breathing region component 108, and the heartbeat region component 109 are indicated.

Also in Embodiment 4, the first threshold 107 of the waveform indicating the motion by shaking of the surface of hot water in the bathtub 142b is 750. Additionally, in Embodiment 4, the second threshold 123 is detected in advance from the amplitude (corresponding to the amplitude of the waveform of the body motion region component) of the signal of the motion component of flowing water discharged from the shower in a state where there is no person (subject) in the bathroom 142.

Specifically, with reference to FIG. 9, in a time 160 with a predetermined length, during which the shower is left running for about 1 minute to 2 minutes after a person exits from the bathroom 142, the signal processing unit 40 detects an average of amplitude values by the motion component of the shower from the waveform of the body motion signal detected by the microwave sensor 140a. In FIG. 9, a waveform 114a of the heartbeat region component and a waveform 114b of the breathing region component are also indicated in the time 160.

As an example, the signal processing unit 40 detects, as the waveform of the motion component of the shower, the waveform 112 of the body motion region component of FIG. 9 input from the body motion signal detection unit 20, decides an amplitude value of the detected waveform of the motion component of the shower as the second threshold 123 (for example, a value=600), and stores the second threshold 123 in the storage unit 402. By performing the pre-processing and setting the second threshold 123 by the motion component of the shower in advance, even when a subject falls down while taking a shower, the signal processing unit 40 is able to compare the amplitude value of the signal of the body motion region component detected by the body motion signal detection unit 20 to the second threshold 123, output a caution signal on the basis of a result of the comparison, and activate the awaking means and the alarming means by an alarm or the like.

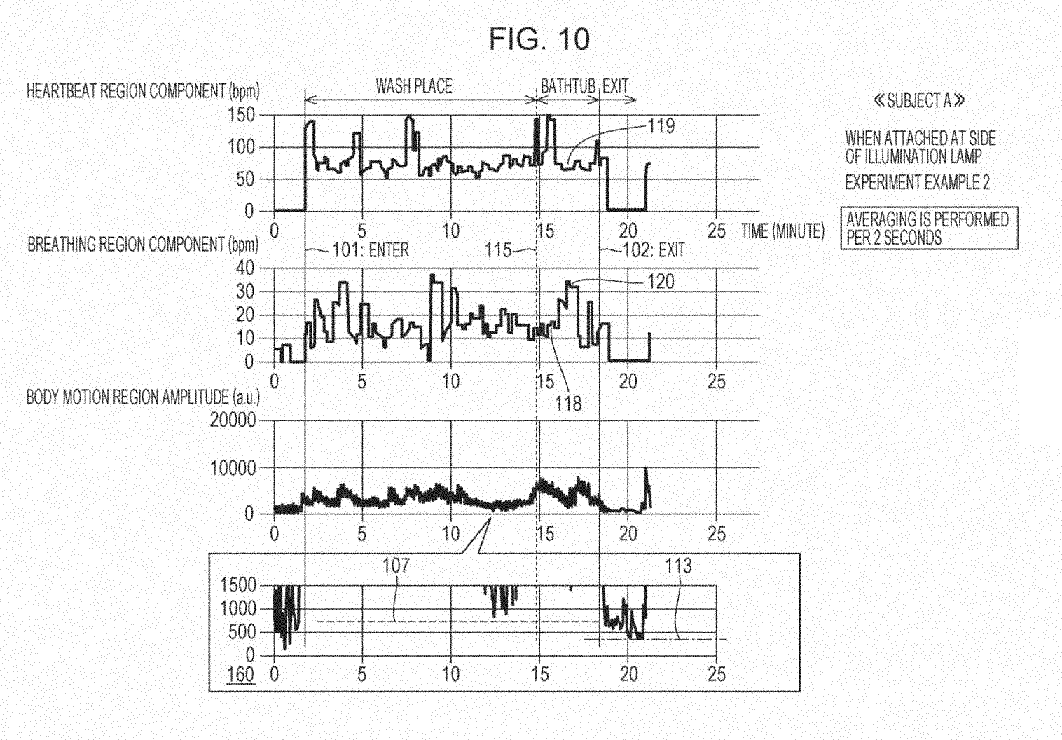

As a specific example, an experiment of the subject A who is a healthy person and does not sleep in the bathtub 142b will be described with reference to FIG. 10. From the time 101 of entrance into the bathroom 142 to the time 115 during which the subject A washes his or her body in the wash place 155 and gets into the bathtub 142b and the time 102 of exit, the amplitude value of the wave by shaking of the surface of hot water in the bathtub 142b exceeds the value of 750 of the first threshold 107 and does not become less than the value of the first threshold 107, and the subject A finishes taking a bath without trouble. Also in this case, when the subject A gets into the bathtub 142b, his or her heart rate and breathing rate are able to be detected as a pulse rate by motion of an artery above a neck and motion of breathing in neck and face portions, and when the subject A remains relatively still in the seating posture, the waveform 119 of the heart rate and the waveform 118 of the breathing rate are roughly able to be detected. When the subject A moves his or her hand while bathing himself or herself in the bathtub 142b, the waveform 120 of the breathing region component is detected and the breathing region component increases.

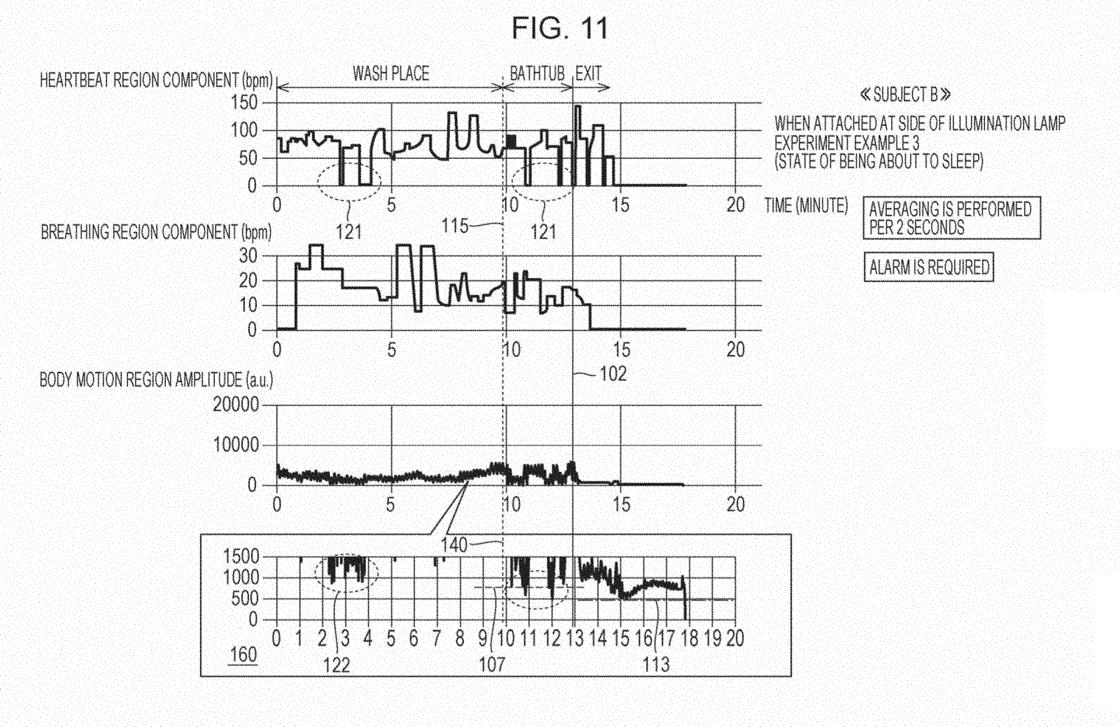

Next, an experiment of the subject B who is a healthy person and falls asleep in the bathtub 142b will be described with reference to FIG. 11. In this experiment, there is a case where, after the time 115 when the subject B gets into hot water in the bathtub 142b, the subject B falls asleep and the amplitude value of the signal of the body motion region component that is detected becomes equal to or less than 750 as the value of the first threshold 107 multiple times repeatedly.

In the case of the subject B, when about two and half minutes have lapsed after start of measurement, even in the wash place 155, an amplitude value 122 of the signal of the body motion region component is small but equal to or more than 750 as the value of the first threshold 107, whereas an amplitude value of a signal 121 of the heartbeat region component falls to zero and there is a situation where the subject B is about to fall asleep. In this case, since the threshold of the signal is set so that the heartbeat becomes zero when the signal of the heartbeat region component has substantially the same level as that of the amplitude of the surface of hot water, but a frequency of the heartbeat region is limited (limited to 3 Hz or less in Embodiment 4), so that there is a case where the heartbeat becomes zero even when the amplitude value of the signal of the heartbeat region component does not have completely the same level as that of the amplitude value of the surface of hot water, and the signal is able to be used as a preliminary caution signal before the first threshold 107 obtained from the signal waveform of the body motion region component is reached.

In a case where the subject B falls asleep in the bathtub 142b, when a case where the amplitude value of the signal of the body motion region component becomes equal to or less than 750 as the value of the first threshold 107 occurs multiple times (for example, three or more times in Embodiment 4) successively, it is possible to output a caution signal and activate the awaking means such as an alarm or the communication means so that the subject is found and saved before he or she completely falls asleep (in a case of an aged person, before falling asleep and drown).

Also in a case where the amplitude value of the signal of the body motion region component is larger than the value (=750) of the first threshold 107 even in the wash place 155, there is a case where the signal of the heartbeat region component intermittently falls to zero, and when such a case occurs multiple times (for example, two or more times in Embodiment 4) successively, the signal processing unit 40 is able to output a caution signal and use the awaking means, such as providing an intermittent alarm signal or making rhythm sound, in order for the subject not to fall asleep, for example.

Similarly to Embodiment 1, it is possible to active the caution signal, the awaking means, and the communication means in a stepwise manner. In Embodiment 4, the amplitude of the signal of the motion component of the shower is set as the second threshold 123, and when a subject falls down while taking a shower, the signal processing unit 40 is able to detect the fall, output the caution signal, and activate the awaking means such as an alarm and the communication means, but the subject A and the subject B finish taking a bath without falling down in the present experiment.

### Embodiment 5

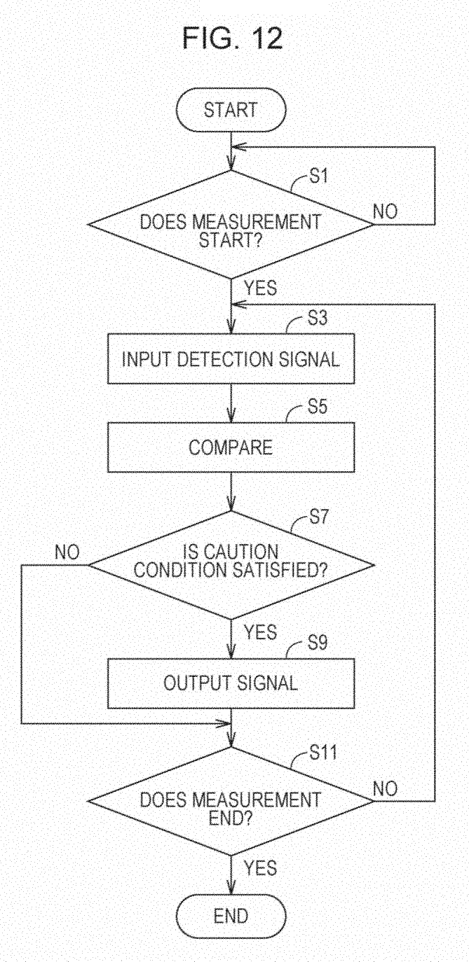

FIG. 12 is a flowchart of processing according to Embodiment 5. The flowchart of the processing is stored in the memory 412 or the recording medium 408 as a program. The CPU 411 reads out the program from such a storage unit and executes the read program. In FIG. 12, it is assumed that the signal processing unit 40 communicates with the body motion signal detection unit 20. The first threshold 107, the second threshold 123, and the noise level 113 that are described in each Embodiment 4 are stored in the memory 412 or the recording medium 408.

With reference to FIG. 12, when the CPU 411 of the body motion signal processing apparatus 100 is activated, the CPU 411 determines whether or not to start measurement (step S1). Specifically, the CPU 411 determines that measurement is to be started when determining that a subject enters the bathroom 142. Here, in the determination of entrance, the CPU 411 compares an amplitude value of a signal of a body motion region component, which is input from the body motion signal detection unit 20, to a predetermined value (for example, the first threshold 107), and when determining, on the basis of a result of the comparison, that the detected amplitude value changes to a large value exceeding the predetermined value, the CPU 411 determines that the subject enters the bathroom 142. Not that, the CPU 411 may determine the entrance on the basis of a sensor output of the door sensor 140C or on the basis of a combination of the body motion signal and the sensor output.

When it is not determined that measurement is to be started (NO at step S1), the processing of step S1 is repeated, but when it is determined that measurement is to be started (YES at step S1), the CPU 411 inputs the signal of the body motion region component detected by the body motion signal detection unit 20 (step S3), detects an amplitude value of the input signal, and compares the detected amplitude value to the first threshold 107 that is stored (step S5). The CPU 411 determines whether or not a condition under which a notice based on a caution signal is to be output is satisfied in a result of the comparison (step 7). For example, when determining that it is detected that a case where the amplitude value of the signal of the body motion region component is the first threshold or less is detected a predetermined number of times on the basis of the result of the comparison in a time period with a predetermined length, the CPU 411 determines that the condition under which a caution signal is to be output is satisfied (YES at step S7). When the CPU 411 determines that the condition is not satisfied (NO at step S7), the processing moves to step S11 described below.

When determining that the condition under which a caution signal is to be output is satisfied (YES at step S7), the CPU 411 outputs a caution signal for a notice through the output control unit 420 to the output unit 90 (step S9). The output unit 90 displays, on the basis of the caution signal, an alarm on the display, outputs alarm sound through the awaking means, or performs transmission to a terminal of a third person through the communication means, and outputs a notice indicating that the subject is in a caution state in the bathroom 142.

Then, the CPU 411 determines whether or not to end the measurement (step S11). Specifically, when determining that the subject exits from the bathroom 142, the CPU 411 determines that the measurement is to be ended. In the determination of exit, the CPU 411 compares an amplitude value of a signal of a body motion region component, which is input from the body motion signal detection unit 20, to a predetermined value (for example, the noise level 113), and when determining, on the basis of a result of the comparison, that the detected amplitude value changes to a value equal to or less than the predetermined value, the CPU 411 determines that the subject exits from the bathroom 142. Note that, the exit may be determined on the basis of an output of the door sensor 140C or on the basis of a combination of the signal of the body motion region component and the sensor output.

When it is determined that the measurement is to be ended (YES at step S11), the processing ends, whereas when it is not determined that the measurement is to be ended (NO at step S11), the processing returns to step S3 and the subsequent processing is repeatedly performed in a similar manner.

### Modified Example

At step S7 of FIG. 12, whether to output a caution signal may be determined in accordance with a combination of the amplitude value of the signal of the body motion region component and the breathing rate or the heart rate as in the conditions (1) to (5) indicated in Embodiment 3. The caution signal may be output by distinguishing the first caution signal and the second caution signal described above. An aspect of outputting an alarm output from the awaking means or content of a transmission signal by the communication means may be different between the first caution signal and the second caution signal.

### Embodiment 6

In Embodiment 6, a program for causing the CPU 411 to execute the processing according to each of the embodiments described above is provided. Such a program is able to be provided also as a program product by being recorded in a computer-readable recording medium attached to a computer of the signal processing unit 40, such as a flexible disk, a CD-ROM (Compact Disk-Read Only Memory), a ROM, a RAM, or a memory card. Alternatively, the program is able to be provided by being recorded in a recording medium such as a hard disk built in a computer. The program is also able to be provided by downloading through a network.

Note that, the program may be the one causing processing to be executed by invoking necessary modules from among program modules provided as a part of an OS (operating system) of a computer, at predetermined timings in a predetermined array. In such a case, the program itself does not include the above modules, and the processing is executed in cooperation with the OS.

Further, the program according to Embodiment 6 may be provided by being incorporated in a part of a different program. Also in such a case, the program itself does not include modules included in the different program, and processing is executed in cooperation with the different program. Such a program incorporated in a different program may also be included in the program according to Embodiment 6.

The provided program product is installed in a program storage unit such as a hard disk, and is executed. Note that, the program product includes the program itself and a recording medium in which the program is recorded.

### [Configuration of Each Embodiment]

The body motion signal processing apparatus 100 includes the signal processing unit 40 that converts a corresponding analog signal of an output signal component from a microwave radar into a digital form and outputs a waveform of body motion that is motion of a body of a person. The body motion signal processing apparatus 100 is attached to a vicinity of the bathroom and has monitoring/alarming/awaking means for motion of the person while the person is taking a bath. The amplitude of only motion of shaking of the surface of hot water in the bathtub 142b is detected in advance, and the amplitude of only the motion is detected and stored in a storage unit. An amplitude level of a signal of a body motion region component by the body motion of the subject 141a in the bathroom 142 is compared to an amplitude level of the motion of the surface of hot water that is stored. Before or when the amplitude level of the signal of the body motion region component of the subject 141a is equivalent to the amplitude level of the motion of the surface of hot water as a result of the comparison, a caution signal is output and the awaking means or the communication means is activated for alarming.

Thus, before or when the amplitude level of the signal of the body motion region component of the subject 141a is almost equivalent to the amplitude level of the motion of the surface of hot water, a caution signal is output, and by the alarming or awaking, the subject is able to be found before being drown.

Further, instead of the amplitude level of the motion by shaking of the surface of hot water, an amplitude level of a signal by motion of flowing water discharged from a shower is able to be used. Thus, before or when the amplitude of the signal of the body motion region component of the subject 141a is equivalent to the amplitude level of the motion of flowing water discharged from the shower, a caution signal is output, and by the awaking or alarming, the subject 141a is able to be found before being drown.

Not only when a sudden change is caused in the amplitude of the signal of the body motion region component but also when a sudden change is caused in a heart rate or a breathing rate, the body motion signal processing apparatus 100 outputs a caution signal. Thus, in addition to a configuration in which monitoring is performed on the basis of body motion, a state of the subject 141a is able to be detected also by a sudden change in the heart rate or the breathing rate, thus making it possible to more reliably and more safely detect whether or not a caution signal is to be output.

The body motion signal processing apparatus 100 is able to be attached to the controller 146 of the hot-water supply system or to the lighting device 145 in the bathroom 142. Thus, power supply to the body motion signal processing apparatus 100 is facilitated.

The signal processing unit 40 detects (calculates) the amplitude of the signal of the body motion region component as average body motion amplitude or moving average body motion amplitude using any absolute value for average time of 0.1 second to 5 seconds. Thus, since the average amplitude of a short time using the absolute value for 0.1 second to 5 seconds is taken, a change in amplitude roughly associated with an action of motion of a person to stand, seat, move, or sleep (loose consciousness) is able to be acquired.

The average body motion amplitude is average body motion amplitude in which the I channel and the Q channel are combined. Since the I channel has cosine body motion amplitude and the Q channel has sine body motion amplitude, a distance between the subject 141a and the body motion signal detection unit 20 (body motion signal processing apparatus 100) causes a zero point every wavelength/2 (for example, about 6 mm in the case of 24 GHz), so that a null point of the cosine and the sine is able to be compensated by combining both the channels by obtaining a sum of root-mean-square values of the I channel and the Q channel, thus making it possible to acquire body motion amplitude stably.

The embodiments disclosed herein are to be construed as illustrative and not limitative in all respects. The scope of the invention is indicated by the claims rather than the foregoing description, and is intended to encompass meanings equivalent to the claims and all modifications falling in the scope of the claims.

### REFERENCE SIGNS LIST

20 biological signal detection unit
40 signal processing unit
90 output unit
100 body motion signal processing apparatus

1. A body motion signal processing apparatus comprising:
a transmission unit that transmits a radio wave to a target which includes a bathroom;
a receiving unit that receives the radio wave reflected by the target; and
a detection unit that detects, from a received signal received by the receiving unit, a body motion component signal corresponding to a body motion component that is motion of a body,
wherein
the body motion signal processing apparatus further comprises a signal processing unit,
wherein, in a case where a subject is in the bathroom, the signal processing unit compares amplitude of the body motion component signal detected by the detection unit to amplitude of the body motion component signal that is detected in advance in the bathroom where no subject exists and to amplitude of noise possessed by the detection unit- and outputs a notice based on a comparison result.

2. The body motion signal processing apparatus according to claim 1, wherein
the body motion component includes a heartbeat component that is motion of the body by heartbeat and a breathing component that is motion of the body by breathing, and
the signal processing unit includes
means for detecting a heart rate and a breathing rate respectively from a heartbeat component signal and a breathing component signal of the body motion component signal, and
in a case where the subject is in the bathroom, outputs the notice based on the comparison result of the amplitude, and the heart rate or the breathing rate.

3. The body motion signal processing apparatus according to claim 1, wherein
the body motion component signal detected in advance in the bathroom where no subject exists includes
a signal of a motion component of shaking of a surface of hot water in a bathtub in the bathroom or a signal of a motion component of flowing water discharged from a shower in the bathroom.

4. The body motion signal processing apparatus according to claim 1, wherein
the received signal by the radio wave reflected includes an IQ modulation signal, and
the amplitude of the body motion component signal indicates amplitude of a signal in which signals of an I channel and a Q channel of the received signal are combined.

5. The body motion signal processing apparatus according to claim 1, wherein
the amplitude of the body motion component signal indicates average amplitude detected by performing sampling of the body motion component signal at a predetermined time interval, and
the signal processing unit further outputs the notice when a plurality of average amplitudes is consecutively less than or equal to the amplitude of the body motion component signal that is detected in advance in the bathroom where no subject exists.
